(19) United States
(12) Patent Application Publication
(10) Pub. No.: US 2012/0071878 A1
(43) Pub. Date: Mar. 22, 2012

(54) FRACTURE MANAGEMENT TOOL AND METHOD

(76) Inventor: David Cowin, Windermere, FL (US)

(21) Appl. No.: 13/241,200

(22) Filed: Sep. 22, 2011

Related U.S. Application Data
(60) Provisional application No. 61/385,297, filed on Sep. 22, 2010.

(51) Int. Cl. A61B 17/16

(52) U.S. Cl. 606/80

(57) ABSTRACT

A bone fragment manipulating tool with reversible pulling mechanism generally comprises an elongated shaft defining a shaft diameter and a drill bit at a distal end. An elongated tubular housing having a housing diameter greater than the shaft diameter encloses the shaft. As the shaft axially retracts in the direction of the tubular housing, a radially protruding structure secures the shaft to a chipped fragment of the bone. The tool then manipulates the fragments to their proper orientations. A method of manipulating a fragment of a fractured bone using a tool generally comprises drilling through the bone, deploying a radially protruding structure to secure the shaft to a chipped fragment, manipulating the chipped fragment to a proper orientation with respect to a primary fragment such that the fragments are aligned and joined, undeploying the radially protruding structure, and removing the tool in a locked, slender configuration from the bone.

### RELATED APPLICATIONS

This application relates to, claims priority from, and incorporates herein by reference as if fully set forth U.S. provisional patent application Ser. No. 61/385297 filed on Sep. 22, 2010 and entitled FRACTURE MANAGEMENT TOOL WITH REVERSIBLE PULLING DEVICE.

### BACKGROUND OF THE INVENTION

1. Field of the Invention The present invention relates to a tool and method for manipulating a fragment of a fractured bone during a bone fracture treatment.

2. Description of Prior Art and Related Information

Bones generally comprise two types of osseous tissue: a dense, strong cortical exterior that facilitates main functions such as supporting the body, protecting the organs, and providing levers for movements; and a softer, highly vascular cancellous (spongy) interior that contains the bone marrow. Bone fracture healing is a natural process in which the body facilitates the repair of a fractured bone. It is known in the orthopedic arts that the treatment of bone fractures generally involve aligning and restoring the fractured fragments of the bone to their natural positions, pressing the fragments on opposite sides of the fracture together, and maintaining those positions for a period of time to allow natural regeneration to occur.

Simple fractures are typically treated by holding the bone fragments together using a cast or a splint. On the other hand, more severe fractures, such as comminuted fractures, intramedullary fractures and intertrochanteric fractures, require more extensive surgical procedures to align and hold the bone fragments together. One method involves attaching metal plates to the outer cortical surfaces of the bones and holding the bone fragments in the desired positions using bone screws. In this method, a fragment of a fractured bone on one side of the fracture is secured to another fragment of the bone on the opposite side of the fracture using a long screw, a nail, a pin, or a combination of a pin and a suture. Another method of treating severe fractures involves the use of a cable wrapped around a broken bone to hold the fragments in place. In this type of procedure, also known as the cerclage treatment, the cable is passed through the bone fragments and entirely encircled around the bone tightly in order to hold the fragments together.

There are other surgical procedures currently known in the art to treat such severe fractures. All these procedures share a common need to manipulate and align the bone fragments to their proper positions prior to holding them together for a period of time.

### BRIEF SUMMARY OF THE INVENTION

One preferred embodiment provides a bone fragment manipulating tool with reversible pulling mechanism that drills through a fractured bone, wherein the fractured bone has at least two fragments on opposite sides of a fracture. The bone fragment manipulating tool then secures to the cortex of a first chipped fragment of the fractured bone, manipulates the first chipped fragment to a proper orientation with respect to a second primary fragment of the fractured bone, and positions the first chipped fragment back in its proper place with respect to the second primary fragment.

The bone fragment manipulating tool with reversible pulling mechanism in a first preferred embodiment generally comprises an elongated shaft defining a shaft diameter and a drill bit at a distal end. An elongated tubular housing having a housing diameter greater than the shaft diameter encloses the entire length of the shaft. As such, the shaft is capable of axially retracting in and out of the tubular housing along a longitudinal axis. In the preferred embodiment, as the drill bit is axially retracted in the proximal direction into the tubular housing, the drill bit shoulders abut the edge of the distal end of the tubular housing. The tubular housing further comprises circumferentially equidistant longitudinal slots on its distal end which form longitudinal panels. As further pressure is applied by the drill bit shoulders against the edge of the distal end of the tubular housing, the longitudinal panels of the tubular housing are forced to deploy and spread, or splay, outwardly to form radially protruding petals of equal length that anchor onto the cortex of the first fragment of the fractured bone.

Another preferred embodiment provides a bone fragment manipulating tool with reversible pulling mechanism that drills through a fractured bone, wherein the fractured bone has at least two fragments on opposite sides of a fracture. The bone fragment manipulating tool then secures to a first chipped fragment of the fractured bone, manipulates the first chipped fragment to a proper orientation with respect to a second primary fragment of the fractured bone, and positions the first chipped fragment back in its proper place with respect to the second primary fragment. The bone fragment manipulation tool in this particular embodiment generally comprises an elongated shaft defining a shaft diameter and a drill bit at a distal end, deployable wings on the shaft adjacent to the drill bit, and an elongated tubular housing having a housing diameter greater than the shaft diameter enclosing the entire length of the shaft.

The shaft is capable of axially retracting in and out of the tubular housing along a longitudinal axis. In this particular embodiment, the drill bit has shoulders that are smaller in size than the diameter of the tubular housing, such that the drill bit can be completely enclosed in the tubular housing as it moves in the proximal direction. The tubular housing further comprises circumferentially equidistant longitudinal slots on its distal end. The wings of the shaft are positioned along the longitudinal slots, such that when deployed, the wings of the shaft can slide along the longitudinal slots as the shaft retracts in and out of the tubular housing. As the shaft and the drill bit move to the farthest position in the proximal direction, the proximal edge of the wings become tightly anchored onto the cortex of the first fragment of the fractured bone.

Another preferred embodiment provides a method of manipulating a fragment of a fractured bone using a bone fragment manipulating tool. The method generally comprises drilling through both the primary bone fragment and the chipped bone fragment such that the drill bit is located on an exterior side of the chipped bone fragment, deploying a radially protruding structure to secure the shaft to an exterior side of the first chipped fragment of the fractured bone, manipulating the first chipped fragment of the fractured bone to a proper orientation with respect to a second primary fragment of the fractured bone such that the two fragments are aligned and joined, restoring and securing the first chipped fragment of the fractured bone into its natural position in the second primary fragment of the fractured bone, undeploying the radially protruding structure so as to collapse the tool to its original, slender state, and removing the tool in a locked, slender configuration from the bone.

### BRIEF DESCRIPTION OF THE DRAWINGS

FIG. 1 illustrates a bone fragment manipulating tool with reversible pulling mechanism comprising a shaft enclosed in a tubular housing in an “undeployed” position according to a preferred embodiment.

FIG. 2a illustrates the bone fragment manipulating tool with reversible pulling mechanism in FIG. 1 in a “deployed” position according to a preferred embodiment.

FIG. 2b illustrates a close up view of the distal portion of the bone fragment manipulating tool with reversible pulling mechanism in FIG. 1, prior to being in a deployed position according to a preferred embodiment.

FIG. 2c illustrates a close up view of the distal portion of the bone fragment manipulating tool with reversible pulling mechanism in FIG. 1, in a deployed position according to a preferred embodiment.

FIG. 3 illustrates the bone fragment manipulating tool with reversible pulling mechanism in FIG. 1 during an initial drilling process.

FIG. 4a illustrates the bone fragment manipulating tool with reversible pulling mechanism in FIG. 1 in an undeployed position during a fragment securing process.

FIG. 4b illustrates the bone fragment manipulating tool with reversible pulling mechanism in FIG. 1 in a deployed position during a fragment securing process.

FIG. 5a illustrates a rear perspective view of the bone fragment manipulating tool with reversible pulling mechanism in FIG. 1 during a fragment manipulation process according to a preferred embodiment.

FIG. 5b illustrates a front perspective view of the bone fragment manipulating tool with reversible pulling mechanism in FIG. 1 after the fragments are restored to their proper positions with respect to each other according to a preferred embodiment.

FIG. 5c illustrates a side view of the bone fragment manipulating tool with reversible pulling mechanism in FIG. 1 after the fragments are restored to their proper positions with respect to each other according to a preferred embodiment.

FIG. 5d illustrates a front view of the bone fragment manipulating tool with reversible pulling mechanism in FIG. 1 after the fragments are restored to their proper positions with respect to each other according to a preferred embodiment.

FIG. 6 illustrates a front perspective view of the bone fragment manipulating tool with reversible pulling mechanism in FIG. 1, wherein an anchor is drilled into the fractured bone to permanently hold the bone fragments together.

FIG. 7 illustrates a front perspective view of the bone fragment manipulating tool with reversible pulling mechanism in FIG. 1, wherein an anchor is in place to permanently hold the bone fragments together and the bone fragment manipulating tool is removed from the bone.

FIG. 8 illustrates a front perspective view of the fractured bone with the bone fragment manipulating tool completely removed.

FIG. 9a illustrates a bone fragment manipulating tool with reversible pulling mechanism according to another preferred embodiment.

FIG. 9b illustrates a bone fragment manipulating tool with reversible pulling mechanism in FIG. 9a, wherein the distal portion of the bone fragment manipulating tool is enclosed within the tool.

FIG. 10a illustrates a perspective view of the bone fragment manipulating tool with reversible pulling mechanism in FIG. 9a with undeployed wings according to a preferred embodiment.

FIG. 10b illustrates a perspective view of the bone fragment manipulating tool with reversible pulling mechanism in FIG. 9a with deployed wings according to a preferred embodiment.

FIG. 11 illustrates the bone fragment manipulating tool with reversible pulling mechanism in FIG. 9a during an initial drilling process according to a preferred embodiment.

FIG. 12a illustrates the bone fragment manipulating tool with reversible pulling mechanism in FIG. 9a during a fragment securing process according to a preferred embodiment.

FIG. 12b illustrates the bone fragment manipulating tool with reversible pulling mechanism in FIG. 9a during a fragment rest process according to a preferred embodiment.

FIG. 13 illustrates a preferred method of manipulating a fragment of a fractured bone using a bone fragment manipulating tool with reversible pulling mechanism.

The invention and its various embodiments can now be better understood by turning to the following detailed description wherein illustrated embodiments are described. It is to be expressly understood that the illustrated embodiments set forth as examples and not by way of limitations on the invention as ultimately defined in the claims.

### DETAILED DESCRIPTION OF THE PREFERRED EMBODIMENTS

As used throughout the specification, the term “distal” refers to a location or a direction away from the tubular housing, whereas the term “proximal” refers to a location or a direction toward the tubular housing.

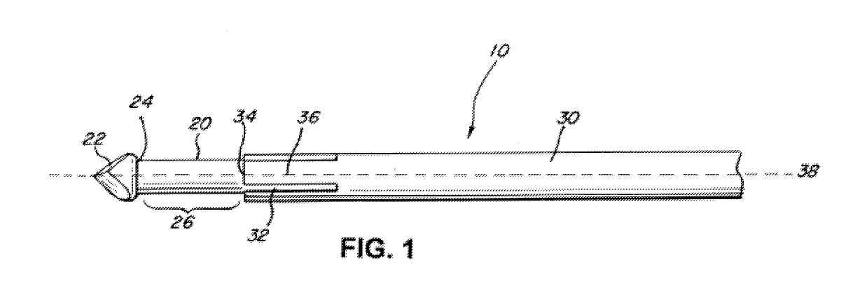

In FIG. 1, a preferred embodiment of a bone fragment manipulating tool with reversible pulling mechanism, or simply bone fragment manipulating tool, is designated by a reference numeral 10. The bone fragment manipulating tool 10 can be used with a suitable power tool or surgical drill (not pictured) for drilling purposes. The bone fragment manipulating tool 10 generally comprises an elongated shaft 20 defining a shaft diameter and a drill bit 22 at a distal end. The drill bit 22 comprises proximal shoulders 24 which protrude radially beyond the shaft diameter. An elongated tubular housing 30 having a housing diameter greater than the shaft diameter encloses the entire length of the shaft 20. As such, the shaft 20 is capable of axially retracting in and out of the tubular housing 30 along a longitudinal axis 38. The bone manipulating tool 10 may comprise such materials as stainless steel, titanium, composite material, cobalt chrome alloy, nitinol or any other suitable materials that preferably have memory characteristics. The list of possible materials is provided for the purpose of illustration and not intended as limiting, it being understood that the selection of materials may depend on the intended application.

Preferably, the drill bit 22 has a shape of an acorn. However, any other suitable shape with similar functionality and performance may be utilized, depending on the particular application. The shaft 20 preferably has a smooth outer surface without any threads to allow smooth rotation of the shaft 20 about the longitudinal axis 38 within the tubular housing 30, as well as smooth retraction of the shaft 20 along the longitudinal axis 38 in and out of the tubular housing 30.

The tubular housing 30 further comprises circumferentially equidistant longitudinal slots 32 on its distal end 34, forming longitudinal panels 36 which remain in an “undeployed” position in its default state. In the illustrated embodiment, the circumferentially equidistant longitudinal slots 32 form at least three longitudinal panels 36. Further, the tubular housing 30 preferably has a smooth outer surface without any threads to allow smooth rotation of the bone fragment manipulating tool 10 about the longitudinal axis 38 within the bone, as well as smooth retraction of the bone fragment manipulating tool 10 along the longitudinal axis 38 in and out of the fractured bone. In this undeployed position, a distance 26 may be created between the drill bit 22 and an edge of the distal end 34 of the tubular housing 30.

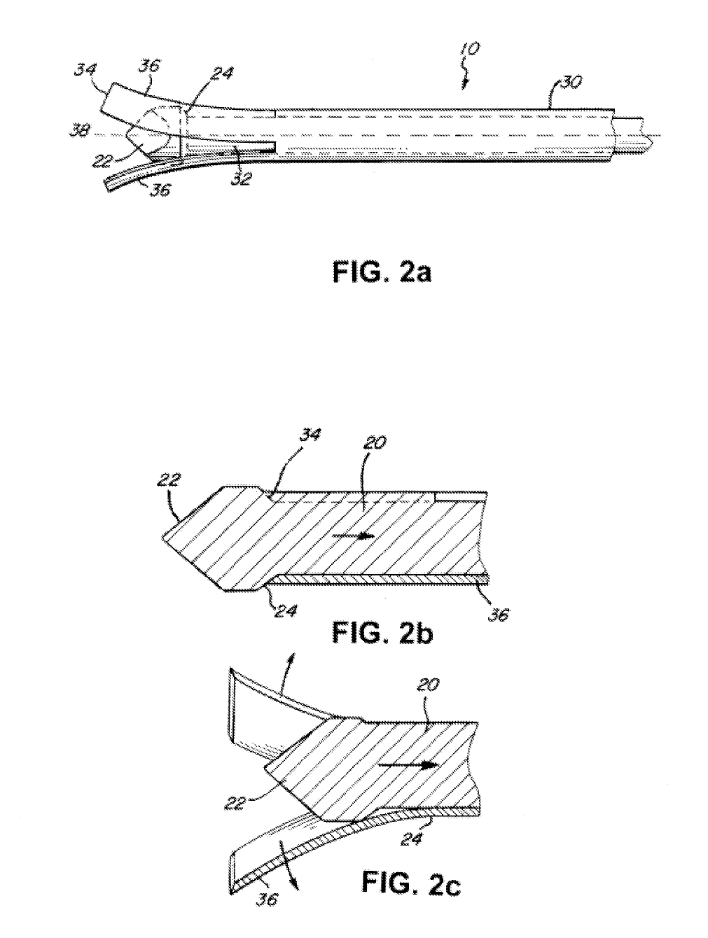

FIGS. 2a-2c illustrate the bone fragment manipulating tool 10 in a “deployed” position. As shown in FIG. 2b, as the drill bit 22 is axially retracted in the proximal direction into the tubular housing 30, the drill bit shoulders 24 abut the edge of the distal end 34 of the tubular housing 30. FIGS. 2a and 2c further demonstrate that as further pressure is applied by the drill bit shoulders 24 against the edge of the distal end 34 of the tubular housing 30, the longitudinal panels 36 of the tubular housing 30 are forced to deploy and spread, or splay, outwardly to form radially protruding petals of equal length. These petals created by the inward force exerted by the drill bit 22 against the inner portion of longitudinal panels 36 function as an anchor that would latch onto the cortex of a bone fragment.

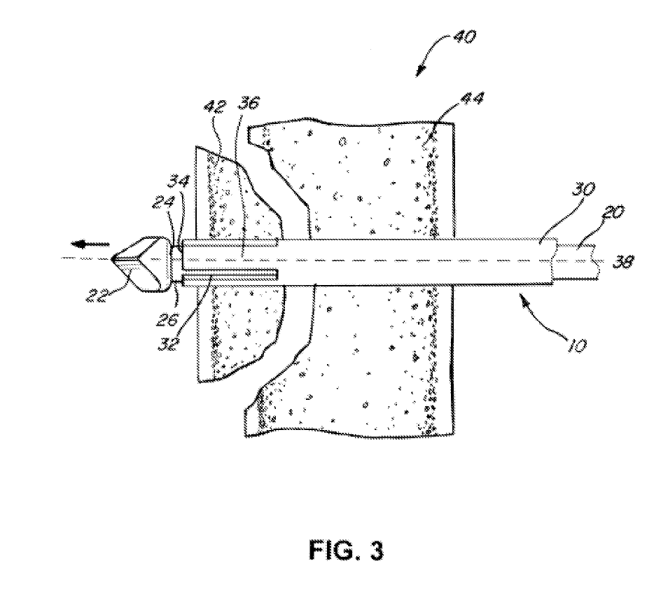

FIG. 3 illustrates a preferred principle of operation of the bone fragment manipulating tool 10 during an initial drilling process. The bone fragment manipulating tool 10 is first drilled through the fractured bone 40 along the longitudinal axis 38, wherein the fractured bone 40 has at least two fragments, a first chipped fragment 42 and a second primary fragment 44, on opposite sides of a fracture. During the drilling process, it is preferable that the bone fragment manipulating tool 10 remains in an undeployed, slim position. As shown in the preferred embodiment, the distance 26 between the drill bit 22 and the edge of the distal end 34 of the tubular housing 30 is maintained through the drilling process. This can be facilitated by a lock mechanism which maintains the shaft 20 in a fixed position with respect to the housing 30. Also shown, the bone fragment manipulating tool 10 is drilled thoroughly past both fragments 42, 44 all the way through the outer side of the first chipped fragment 42.

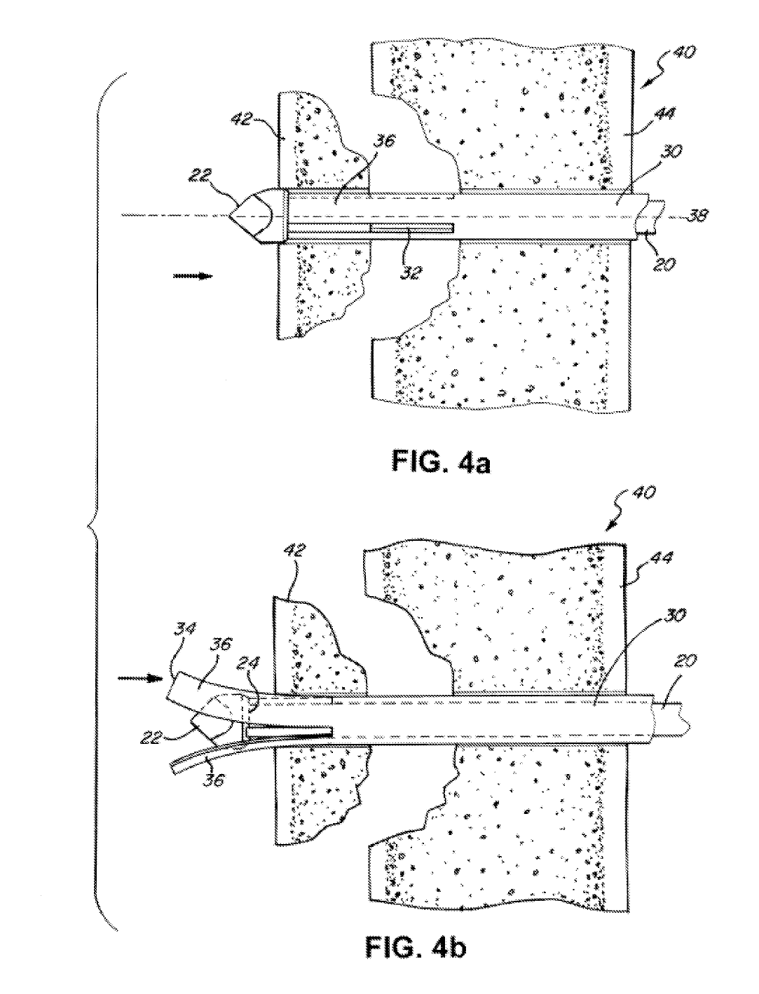

FIGS. 4a-4b illustrate a preferred principle of operation of the bone fragment manipulating tool 10 during a fragment securing process. Subsequent to the drilling process, the drill bit 22 of the elongated shaft 20 and the longitudinal panels 36 of the tubular housing 30 are now on the outer side 48 of the first chipped fragment 42 of the fractured bone 40. The lock mechanism is now unlocked in order to allow movement of the shaft 20 with respect to the housing 30. The drill bit 22 is then retracted in the proximal direction along the longitudinal axis 38 into the tubular housing 30. As shown in FIG. 4a, as the drill bit 22 moves proximally, the drill bit shoulders 24 abut the edge of the distal end 34 of the tubular housing 30. FIG. 4b illustrates that as the drill bit shoulders 24 are retracted further proximally, the shoulders 24 apply force against the inner walls of the longitudinal panels 36 of the tubular housing 30, forcing the panels 36 to spread out into petals, preferably of equal length. The rigidity of the longitudinal panels 36 of the tubular housing 30 and the tension created by pulling the drill bit 22 inward against the inner walls of the longitudinal panels 36 maintain the petal shape that functions as a tight anchor onto the cortex of the first chipped fragment 42 of the fractured bone 40. The first chipped fragment 42 of the fractured bone 40 is now secured by the bone fragment manipulating tool 10.

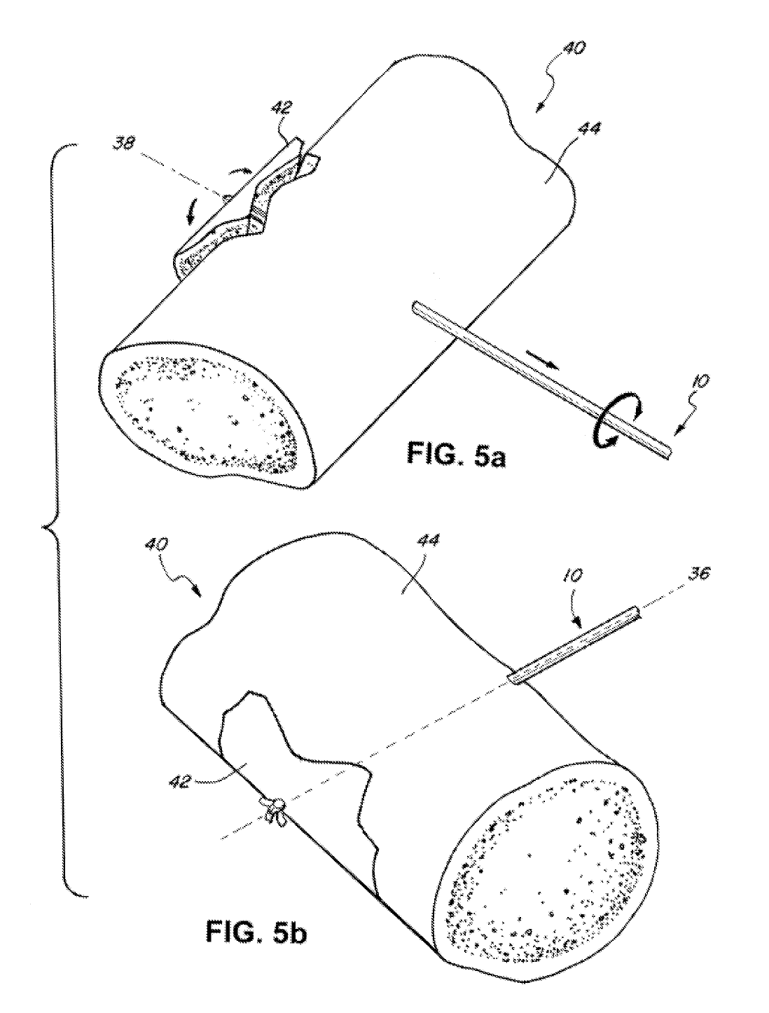

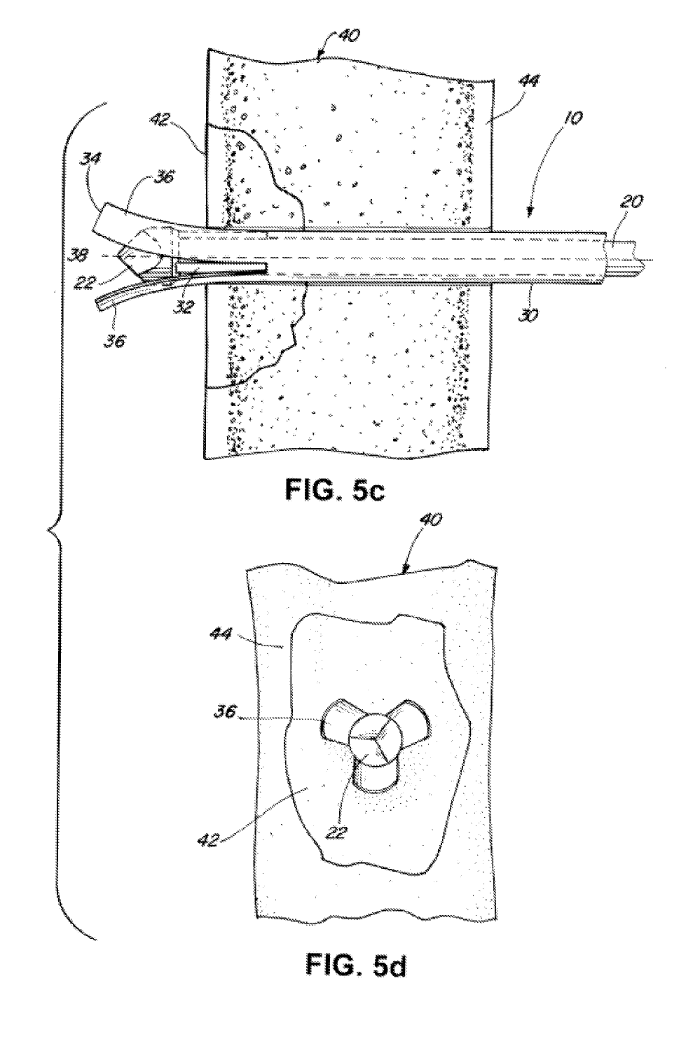

FIG. 5a illustrates a rear perspective view of a preferred principle of operation of the bone fragment manipulating tool 10 during a fragment manipulation process. As shown in FIG. 5a, the elongated shaft 20 and the elongated tubular housing 130 are rotatable as a unit about the longitudinal axis 38 and retractable as a unit in and out of the bone. After the first chipped fragment 42 of the fractured bone 40 is secured, the first chipped fragment 42 is manipulated to a proper orientation with respect to the second primary fragment 44 of the fractured bone 40 by rotating the bone fragment manipulating tool 10 clockwise or counterclockwise about the longitudinal axis 38.

FIGS. 5b and 5c illustrate a front perspective view and a side view showing that after the first chipped fragment 42 and the second primary fragment 44 of the fractured bone 40 are properly aligned with respect to each other along the fracture, the first chipped fragment 42 of the fractured bone is restored to its proper position with respect to the second primary fragment 44 of the fractured bone 40 by manipulating the bone fragment manipulating tool 10 inwardly or proximally toward the second bone fragment 44. Pressure is maintained to ensure that the two fragments 42, 44 are held together in place, while a permanent anchor such as a long screw, a nail, a pin, or a combination of a pin and a suture is placed into the fractured bone 40.

FIG. 5d illustrates a front view of the bone fragment manipulating tool 10, wherein the longitudinal panels 36 are tightly anchored to the cortex of the first chipped fragment 42, and the first chipped fragment 42 and the second primary fragment 44 are held together.

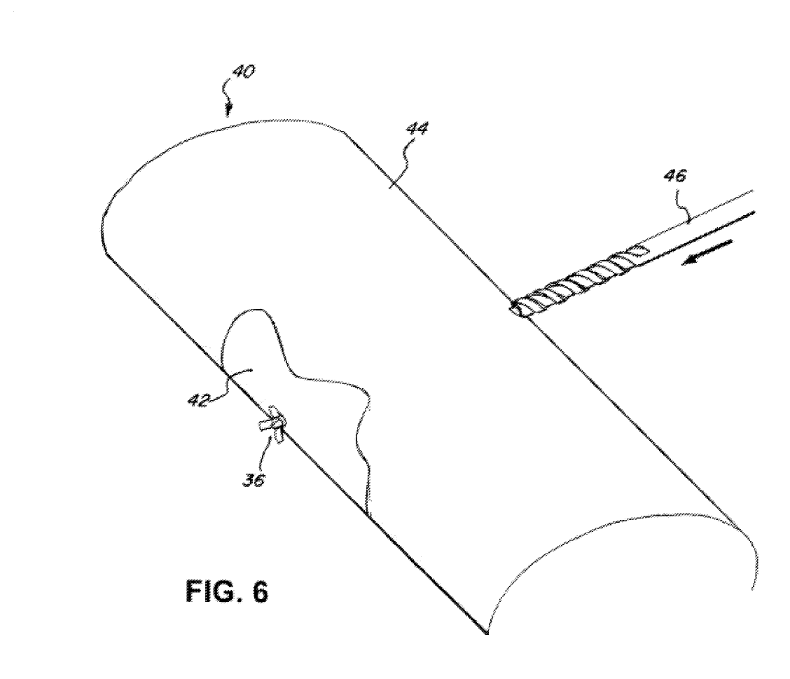

In FIG. 6, while the bone fragment manipulating tool 10 holds the first chipped fragment 42 and the second primary fragment 44 together, an appropriate securing mechanism, such as a surgical drill over-screw 46 which is threaded over the shaft 20 and inserted into the fractured bone 40 to permanently anchor the bone fragments together.

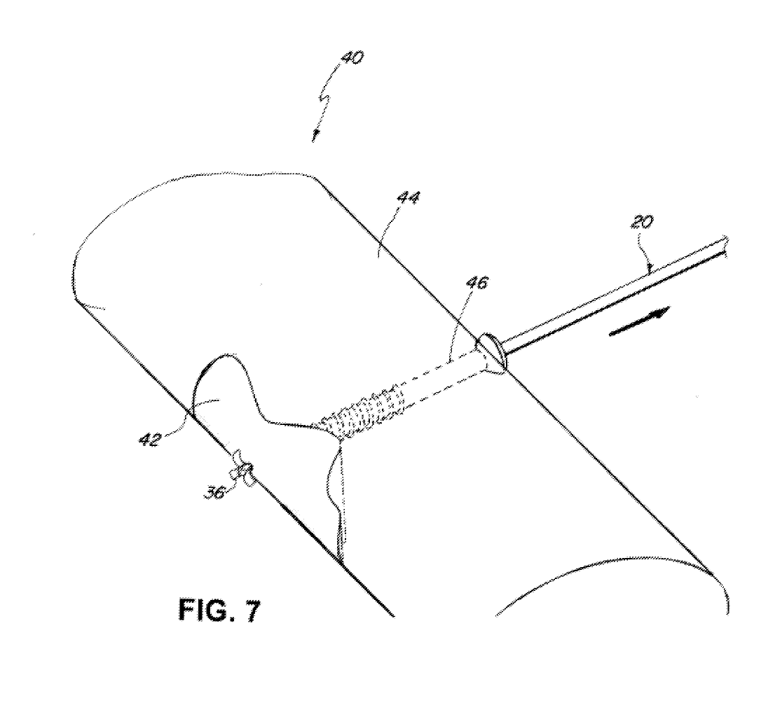

In FIG. 7, after the first chipped fragment 42 and the second primary fragment 44 of the fractured bone 40 are permanently secured using the surgical drill screw 46, the bone fragment manipulating tool 10 is removed by pushing the shaft 20 distally with respect to the housing 30 to restore the longitudinal panels 36 of the tubular housing 30 to their slender, undeployed position. The housing 30 preferably comprises a flexible material having memory characteristics such that the longitudinal panels 36 return to its original slender cylindrical configuration when the drill bit 22 is pushed out of the housing 30. The shaft 20 is then locked to the housing 30 so that the entire tool 10 can be pulled out of the entire bone fragment while maintaining the slender, undeployed configuration.

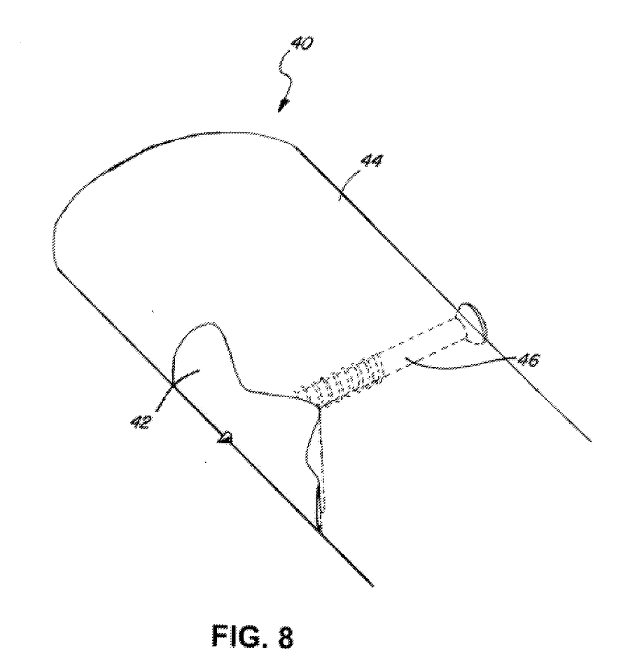

FIG. 8 illustrates the first chipped fragment 42 and the second primary fragment 44 of the fractured bone 40 permanently secured using the surgical drill screw 46, with the bone fragment manipulating tool completely removed.

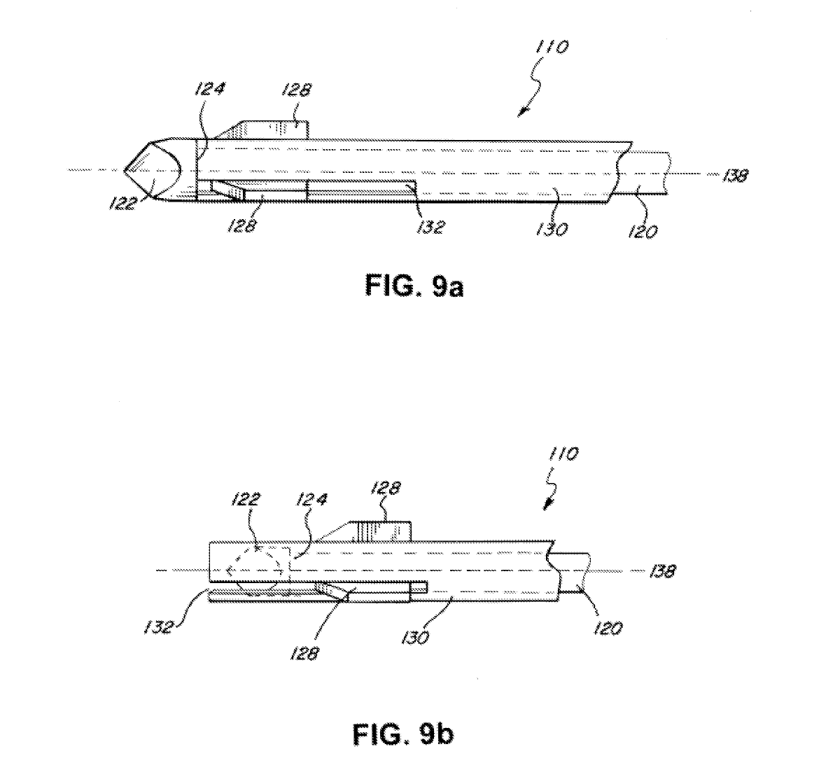

FIG. 9a illustrates a second preferred embodiment of the bone fragment manipulating tool designated by a reference numeral 110. The bone fragment manipulating tool 110 in this particular embodiment generally comprises a drill bit 122 on its distal end, an elongated shaft 120 having a shaft diameter, deployable wings 128 adjacent to the drill bit 122 on the shaft 120, and an elongated tubular housing 130 having a housing diameter greater than the shaft diameter and enclosing the entire length of the shaft 120. In this preferred embodiment, the elongated shaft 120 comprises at least three deployable wings 128. The deployable wings 128 each may comprise a semi-trapezoidal shape, rectangular shape, square shape, or any other suitable shape with similar functionality and performance, depending on the particular application. Further, the axial movement of the deployable wings 128 in and out of the tubular housing 130 can be facilitated by a spring mechanism that allows the deployable wings 128 to collapse as the shaft 120 retracts proximally into the tubular housing 130. Further, FIG. 9b shows the drill bit 122 preferably having shoulders 124 that are smaller in size than the diameter of the tubular housing 130, such that the drill bit 122 can be completely enclosed in the tubular housing 130 as it axially moves in the proximal direction.

The bone manipulating tool 10 may comprise such materials as stainless steel, titanium, composite material, cobalt chrome alloy, nitinol or any other suitable materials that preferably have memory characteristics. The lists of possible shapes of the wings 128 and possible materials of the bone manipulating tool 110 are provided for the purpose of illustration and not intended as limiting, it being understood that the selection of materials may depend on the intended application.

In this embodiment, the shaft 120 has a smooth outer surface without any threads to allow smooth rotation of the shaft 120 about the longitudinal axis within the tubular housing 130, as well as smooth retraction of the shaft 120 along the longitudinal axis in and out of tubular housing 130. The tubular housing 130 also has a smooth outer surface without any threads to allow smooth rotation of the bone fragment manipulating tool 110 about the longitudinal axis within the fractured bone, as well as smooth retraction of the bone fragment manipulating tool along the longitudinal axis in and out of the fractured bone. FIGS. 9a-9b demonstrate the tubular housing 130 further comprising circumferentially equidistant longitudinal slots 132 on its distal end.

The wings 128 of the shaft are positioned along the longitudinal slots 132, such that when deployed, the wings 128 slide along the longitudinal slots 132 as the shaft 120 retracts in and out of the tubular housing 130 along the longitudinal axis 138. As the drill bit 122 moves to the farthest position in the proximal direction, the proximal edge of the wings 128 abut the proximal edge of the longitudinal slots 132.

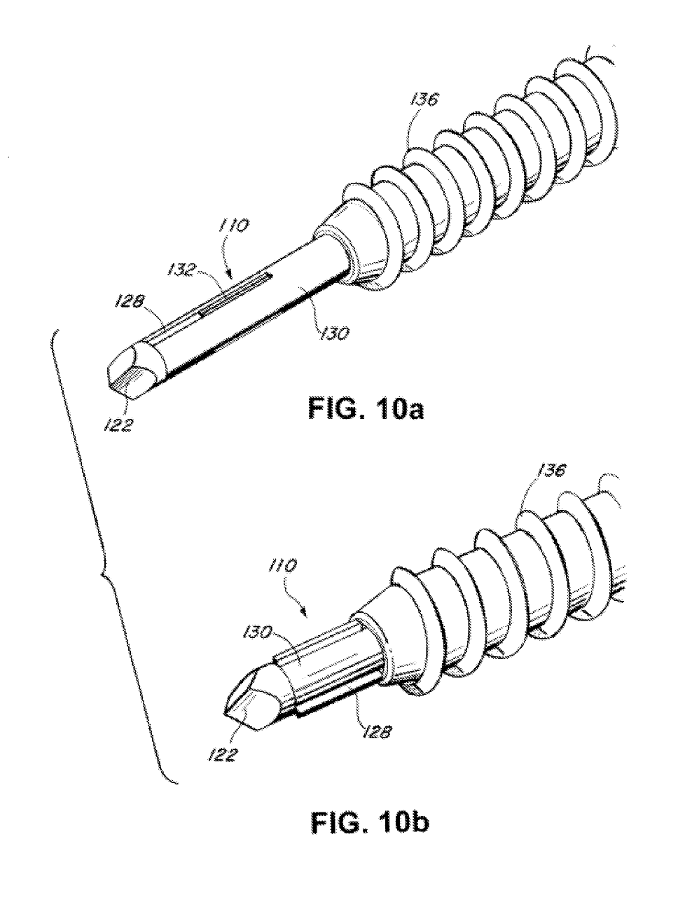

FIGS. 10a-10b illustrate a perspective view of the bone fragment manipulating tool 110. In FIG. 10a, the shaft 120 and the wings 128 are completely enclosed within the tubular housing during an initial drilling process. Additionally, the tool 110 may be coupled to other commercially available equipment, such as a surgical drilling screw 136. As shown in FIG. 10b, the entire bone fragment manipulating tool 110 can slide in and out of the surgical drilling screw 136. During the drilling process, it is preferable that the deployable wings remain in an undeployed, slim position. This can be facilitated by a lock mechanism which maintains the bone fragment manipulating tool 110 in a fixed position with respect to the housing drilling screw 136. Once the bone fragments are held together, the surgical drilling screw 136 can remain in the fractured bone to hold the fragments together, while the bone manipulating tool 110 is removed from the bone.

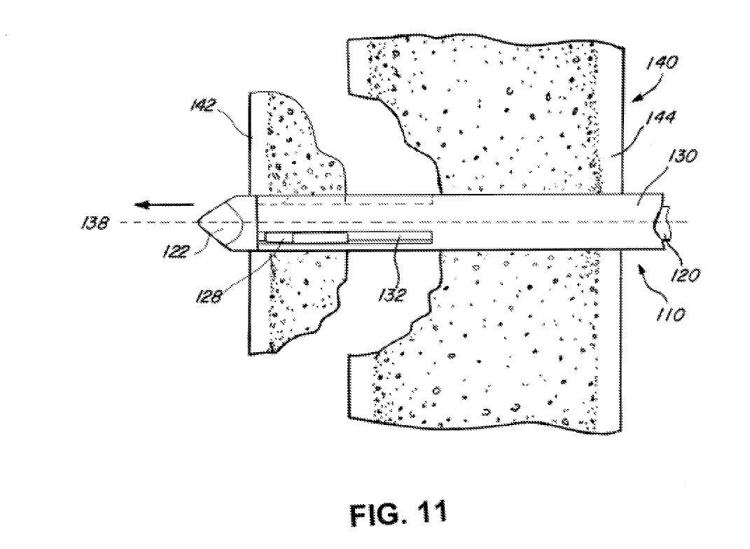

FIG. 11 illustrates a preferred principle of operation of the bone fragment manipulating tool 110 during an initial drilling process. The bone fragment manipulating tool 110 is first drilled through the fractured bone 140 along a longitudinal axis 138, wherein the fractured bone 140 has at least two fragments, a first chipped fragment 142 and a second primary fragment 144, on opposite sides of a fracture. During the drilling process, the bone fragment manipulating tool 110 remains in an undeployed slender configuration, wherein the deployable wings 128 are still enclosed within the tubular housing 130. Also shown, the bone fragment manipulating tool 110 is drilled thoroughly past both fragments 142, 144 all the way through the outer side of the first chipped fragment 142.

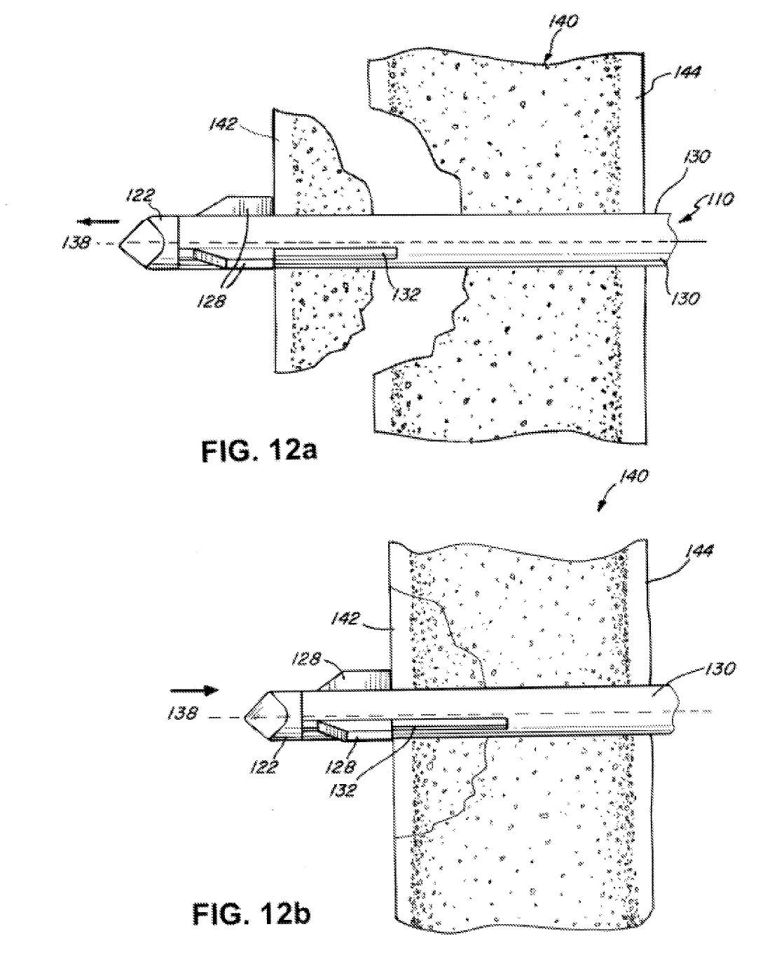

FIGS. 12a-12b illustrate a preferred principle of operation of the bone fragment manipulating tool 110 during a fragment securing process. FIG. 12a shows that subsequent to the drilling process, the drill bit 122 of the shaft 120 and part of the tubular housing 130 which encloses the wings 128 are now on the outer side 148 of the first chipped fragment 142 of the fractured bone 140. As the drill bit 122 moves in the distal direction along the longitudinal axis 138, the deployable wings 128 are deployed. In FIG. 12b, the drill bit 122 is then retracted in the proximal direction into the tubular housing 130. The deployable wings 128 are pressed against the cortex of the first chipped fragment 142 of the fractured bone 140. Upon further pressure in the proximal direction, the deployable wings 128 apply force against the cortex of the first chipped fragment 142 of the fractured bone 140 as a tight anchor. The first chipped fragment 142 of the fractured bone 140 is now secured by the bone fragment manipulating tool 110.

The bone manipulating tool 110 has a similar principle of operation as the bone manipulating tool 10 in the first preferred embodiment during a fragment manipulation process. See FIGS. 5a-5b, and 6-8.

In the preferred embodiments of the tool discussed above, it will be appreciated that each embodiment includes a deployable anchor configured to be secured to the outer side of a chipped bone fragment. The deployable anchor comprises structures which protrude radially outwardly as to secure the external side of the chipped bone fragment. Each embodiment comprises a shaft retractable with respect to an outer tube housing so as to deploy (e.g., protrude outwardly) and un-deploy (e.g., return to original, slender configuration) the deployable anchors. Thus, each preferred embodiment of the tool is configured to have an original slender configuration for drilling, spread out to a temporary flared out configuration of the anchor for securing and manipulating the chipped bone fragment, and to return to a slender profile for removing the tool from the bone assembly.

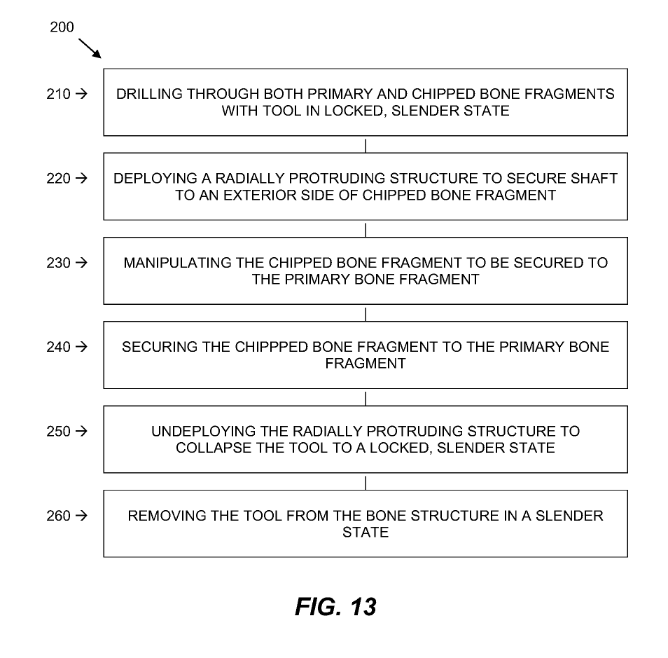

FIG. 13 illustrates a preferred method 200 of manipulating a fragment of a fractured bone using a bone fragment manipulating tool, wherein the tool comprises a drill bit formed on a distal tip of an elongated shaft that is axially movable with respect to a tubular housing in which the shaft is enclosed. The shaft is movable with respect to the housing in order to deploy and undeploy radially protruding structures which secure to the exterior side of the chipped bone fragment.

The method 200 comprises the step 210 of drilling through both the primary bone fragment and the chipped bone fragment such that the drill bit is located on an exterior side of the chipped bone fragment. Step 210 comprises maintaining the shaft in a locked position with respect to the tubular housing. Step 210 also comprises maintaining the entire tool in a slender, undeployed configuration to facilitate drilling through bone matter.

Step 220 generally comprises deploying a radially protruding structure to secure the shaft to an exterior side of the first chipped fragment of the fractured bone. Step 220 comprises retracting unlocking the tool to enable the shaft to move with respect to the housing. In one embodiment, step 220 may comprise retracting a shaft having proximal shoulders in the proximal direction with respect to the tubular housing having distal slits so as to form longitudinal panels. This dynamic causes the drill bit shoulders to press against the inner walls of longitudinal panels to spread out into petals of equal length. The shaft is further retracted inward to tightly anchor the petals onto the cortex of the first chipped fragment of the fractured bone.

Alternatively, step 220 may include providing a shaft which further comprises deployable wings adjacent to the drill bit, providing a tubular housing which further comprises circumferentially equidistant longitudinal slots on its distal end, and positioning the wings along the longitudinal slots, such that when deployed, the wings slide along the longitudinal slots as the shaft retracts in and out of the tubular housing. In this alternative step, subsequent to drilling the bone fragments, the wings are deployed and pressed against the cortex of the first chipped fragment of the fractured bone, and the shaft and the drill bit are retracted into the tubular housing to tightly anchor the wings onto the cortex of the first chipped fragment of the fractured bone.

Step 230 comprises manipulating the first chipped fragment of the fractured bone to a proper orientation with respect to the second primary fragment of the fractured bone such that the two fragments are aligned and joined. This step may include rotating the bone manipulating tool clockwise or counterclockwise about its longitudinal axis until a proper orientation of the bone fragments with respect to each other is achieved and/or moving the chipped fragment toward the primary bone fragment.

Step 240 comprises restoring and securing the first chipped fragment of the fractured bone into its natural position in the second primary fragment of the fractured bone. This step 240 may include retracting the bone manipulating tool inward into the bone after a proper alignment is achieved, maintaining pressure to ensure that the bone fragments are held together in place while employing a permanent anchor such as a long screw, a nail, a pin, or a combination of a pin and a suture is placed into the fractured bone. This step 240 may also comprise deploying an over-screw anchor threaded over the tool.

Step 250 comprises undeploying the radially protruding structure so as to collapse the tool to its original, slender state. In one embodiment, this step 250 comprises unlocking the tool and pushing the shaft distally with respect to the housing with petals to remove the force applied to the petals and, thus, enable the petals to return to its default, slender configuration. The shaft can then be locked to the housing to maintain a fixed, slender configuration of the tool so that the tool is ready to be removed from the bone. In a second preferred embodiment, this step 250 comprises retracting wings back into the housing, and then locking the tool in this slender configuration.

Step 260 comprises removing the tool in a locked, slender configuration from the bone.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements. The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A bone fragment manipulating tool comprising:
an elongated shaft having a drill bit on its distal end and a shaft diameter;
an elongated tubular housing enclosing the shaft and having a housing diameter greater than the shaft diameter; and
circumferentially equidistant longitudinal slots on the distal end of the tubular housing which form longitudinal panels,
wherein the shaft is retractable in and out of the tubular housing along a longitudinal axis, and
wherein the longitudinal panels spread outward to form petals when the shaft is retracted into the housing, and return to an original slender state when the shaft is pushed distally from the housing.

2. The tool of claim 1, wherein the longitudinal slots on the distal end of the tubular housing form at least three longitudinal panels.

3. The tool of claim 1, wherein the shaft and the tubular housing are rotatable as a unit about the longitudinal axis.

4. The tool of claim 1, wherein the shaft and the tubular housing are retractable as a unit along the longitudinal axis.

5. The tool of claim 1, wherein the tubular housing has a smooth outer surface without any threads to allow smooth rotation of the tool about the longitudinal axis within the bone, as well as smooth retraction of the tool along the longitudinal axis in and out of the bone.

6. The tool of claim 1, wherein the shaft has a smooth outer surface without any threads to allow smooth rotation of the shaft about the longitudinal axis within the tubular housing, as well as smooth retraction of the shaft along the longitudinal axis in and out of the tubular housing.

7. The tool of claim 1, wherein the drill bit has a shape of an acorn.

8. A bone fragment manipulating tool comprising:
an elongated shaft having a drill bit on its distal end and a shaft diameter;
deployable wings adjacent to the drill bit on the shaft;
an elongated tubular housing enclosing the shaft and having a housing diameter greater than the shaft diameter; and
circumferentially equidistant longitudinal slots on the distal end of the tubular housing,
wherein the shaft is retractable in and out of the tubular housing along a longitudinal axis, the deployable wings are positioned along the longitudinal slots, the deployable wings slide along the longitudinal slots and anchor onto a cortex of a bone fragment upon retracting the shaft and the drill bit in the direction of the tubular housing, and the deployable wings return to an original slender state when the shaft is pushed distally from the housing.

9. The tool of claim 8, wherein the shaft further comprises at least three deployable wings.

10. The tool of claim 8, wherein the shaft and the tubular housing are rotatable as a unit about the longitudinal axis.

11. The tool of claim 8, wherein the shaft and the tubular housing are retractable as a unit along the longitudinal axis.

12. The tool of claim 8, wherein the tubular housing has a smooth outer surface without any threads to allow smooth rotation of the tool about the longitudinal axis within the bone, as well as smooth retraction of the tool along the longitudinal axis in and out of the bone.

13. The tool of claim 8, wherein the shaft has a smooth outer surface without any threads to allow smooth rotation of the shaft about the longitudinal axis within the tubular housing, as well as smooth retraction of the shaft along the longitudinal axis in and out of the tubular housing.

14. A method of manipulating a fragment of a fractured bone using a bone fragment manipulating tool with reversible pulling mechanism, wherein the tool comprises a drill bit formed on a distal tip of an elongated shaft that is axially movable with respect to a tubular housing in which the shaft is enclosed, comprising:
drilling through both a secondary primary bone fragment and a first chipped bone fragment such that the drill bit is located on an exterior side of the first chipped bone fragment;
deploying a radially protruding structure to secure the shaft to an exterior side of the first chipped fragment of the fractured bone;
manipulating the first chipped fragment of the fractured bone to a proper orientation with respect to the second primary fragment of the fractured bone such that the two fragments are aligned and joined;
restoring and securing the first chipped fragment of the fractured bone into its natural position in the second primary fragment of the fractured bone;
undeploying the radially protruding structure so as to collapse the tool to its original, slender state; and
removing the tool in a locked, slender configuration from the bone.

15. The method of claim 14, wherein drilling through both a secondary primary bone fragment and a first chipped bone fragment such that the drill bit is located on an exterior side of the first chipped bone fragment further comprises maintaining the shaft in a locked position with respect to the tubular housing.

16. The method of claim 14, wherein deploying a radially protruding structure to secure the shaft to an exterior side of the first chipped fragment of the fractured bone further comprises retracting a shaft having proximal shoulders in the proximal direction with respect to the tubular housing having distal slits so as to form longitudinal panels.

17. The method of claim 14, wherein manipulating the first chipped fragment of the fractured bone to a proper orientation with respect to the second primary fragment of the fractured bone such that the two fragments are aligned and joined further comprises rotating the bone manipulating tool clockwise or counterclockwise about its longitudinal axis until a proper orientation of the bone fragments with respect to each other is achieved and/or moving the chipped fragment toward the primary bone fragment.

18. The method of claim 14, wherein restoring and securing the first chipped fragment of the fractured bone into its natural position in the second primary fragment of the fractured bone further comprises retracting the bone manipulating tool inward into the bone after a proper alignment is achieved, maintaining pressure to ensure that the bone fragments are held together in place while employing a permanent anchor such as a long screw, a nail, a pin, or a combination of a pin and a suture is placed into the fractured bone.

19. The method of claim 14, wherein undeploying the radially protruding structure so as to collapse the tool to its original, slender state further comprises unlocking the tool and pushing the shaft distally with respect to the housing.

20. The method of claim 14, wherein deploying a radially protruding structure to secure the shaft to an exterior side of the first chipped fragment of the fractured bone further comprises providing a shaft which further comprises deployable wings adjacent to the drill bit, providing a tubular housing which further comprises circumferentially equidistant longitudinal slots on its distal end, and positioning the wings along the longitudinal slots.
